(19) United States
(12) Patent Application Publication
(10) Pub. No.: US 2006/0243273 A1
(43) Pub. Date: Nov. 2, 2006

(54) Dispensing device

(76) Inventor: Andrew David Wright, Norfolk (GB)

Correspondence Address: MORRIS MANNING MARTIN LLP, 3343 PEACHTREE ROAD, NE, 1600 ATLANTA FINANCIAL CENTER, ATLANTA, GA, 30326, US

(21) Appl. No.: 10/502,231

(22) PCT Filed: Jan. 23, 2003
(86) PCT No.: PCT/GB03/00287

§ 371 Date: Mar. 25, 2005

(30) Foreign Application Priority Data
Jan. 23, 2002 (GB) 0201507.1

(51) Int. Cl. A61M 11/00

(52) U.S. Cl. 128/200.14

(57) ABSTRACT

The present invention relates to a method of dispensing a liquid suspension from a reservoir of a liquid suspension held in a delivery system, wherein the reservoir of liquid suspension is of a type which is normally isolated from atmosphere, comprising the steps of: a) increasing the volume of the reservoir above an initial volume so as to reduce the pressure in the reservoir to below atmospheric; b) agitating the liquid suspension; c) reducing the volume of the reservoir to the initial volume; and d) subsequently dispensing at least a portion of the liquid suspension from the reservoir.

The invention relates to dispensing devices and, particularly to dispensing devices for dispensing medicaments in suspension in a spray form.

Delivery systems are known for administering liquids in spray form. One such system is described in published application WO91/15303. A problem has been found with dispensing liquids, including medicaments, which are formed from suspensions using such devices. It is a requirement of such devices that the liquid medicament is stored without any air being present in the medicament cartridge or medicament vial. This is due to the fact that the air can cause degradation of the medicament, because of the dangers of injecting air into the patient, especially where the medicament is used intravenously, and also the inconsistency in shot weight which occurs when air is present. As a result, the vials or cartridges or other containers for the medicament are formed with no air space. A problem has been found with dispensing suspensions from such vials or cartridges since it has been found that over time, the suspended element of the suspension settles out from the liquid carrier. In order for a correct dosage to be dispensed, it is therefore necessary to agitate the medicament to resuspend the medicament in the liquid carrier. This is not easy to achieve efficiently with many suspensions in a medicament vial or cartridge where there is no air space or other head space.

The present invention provides a method of dispensing a liquid suspension from a reservoir of a liquid suspension held in a delivery system, wherein the reservoir of liquid suspension is of the type which is normally isolated from atmosphere, comprising the steps of:
a) increasing the volume of the reservoir above an initial volume so as to reduce the pressure in the reservoir to below atmospheric;
b) agitating the liquid suspension;
c) reducing the volume of the reservoir to the initial volume; and
d) subsequently dispensing at least a portion of the liquid suspension from the reservoir.

The present invention also provides a delivery system for dispensing a liquid suspension comprising a reservoir of the liquid suspension of the type which is normally isolated from atmosphere, means for creating at least a partial vacuum in the reservoir of liquid suspension and means for dispensing at least a portion of the liquid from the reservoir subsequent to agitation of the liquid suspension.

The present invention also provides use of the delivery system.

An embodiment of the present invention will now be described, by way of example only, with reference to the accompanying drawing, which is a sectional elevation through a dispensing device according to the invention.

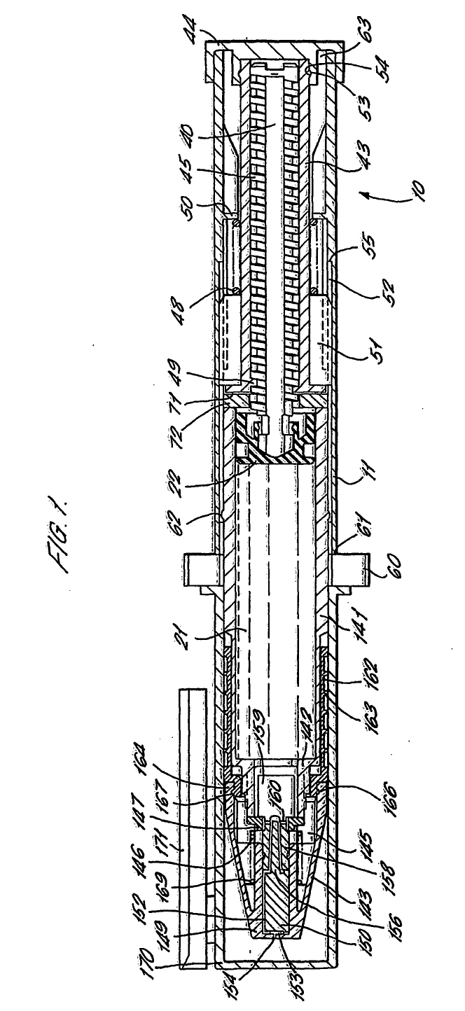

Referring to FIG. 1, there is illustrated a dispensing device intended for dispensing a liquid medicament in the form of a spray. The device forms a delivery system which is particularly suitable for nasal administration of a medicament.

The dispensing device comprises a drive unit 10, a dispenser housing 11, a nozzle 143 and a protective cap 170.

The dispenser housing 11 is a cylindrical tube. A generally cylindrical housing 141 is received slidingly inside, and connected to, the dispenser housing 11 by means of a snap-fit co-operating feature 62. The generally cylindrical housing 141 has a stepped end 142 which is generally closed off and connects to the nozzle 143 of the device and includes at its other end a piston 22 which is sealingly received in the cartridge 141 so as to isolate the contents of the cartridge 141 from atmosphere when the nozzle 143 is in the closed position. The drive unit 10 is arranged to move the piston 22 to the left as viewed in FIG. 1 by pre-selected distances corresponding to pre-selected doses of medicament stored in the device which are to be dispensed. The operation of the drive unit 10 will be described further below.

The stepped end 142 of the generally cylindrical housing 141, which serves as a medicament cartridge, is closed off by a valve cap 145 having a central aperture 146 fixed to the end of the cartridge 141 and trapping a valve seat 147 between the valve cap 145 and the open end of stepped portion 142 of the cartridge 141.

The nozzle 143 of the device comprises outer and inner nested components 149 and 150 which define between them a flow path for medicament to be dispensed. The nozzle 143 is of the type designed to dispense a pressurised liquid passing through the nozzle in the form of a spray. The construction of the nozzle 143 may be, for example, as described in published European patent application 0 308 100. In summary, an axially extending channel 152 is defined between the inner and outer components 149, 150 leading to a swirl chamber 153 and outlet orifice 154 of the device. The inner end of the inner nested component 150 includes a tapered peg 156 which is fitted in a tapered bore of a valve core 158. The valve core 158 includes an enlarged portion 159, the shoulder of which engages the valve seat 147 to isolate the contents of cartridge 141 from the atmosphere when the device is not in use.

When the portion 159 is unseated from valve seat 147, medicament may flow into the nozzle 143 through side holes 160 in the valve core 158 and thence through a flow path formed between tapered peg 156 and the bore in which it seats. This connects in turn with axial bore 152 in nozzle 143.

The inner and outer nested components 149, 150 of the nozzle 143 and the valve core 158 are fixed together to move as a single component. The assembly of nozzle and valve core is moved by a slider 162 including finger grips 163 which is fixed to a skirt portion 164 of the outer component 149 of the nozzle assembly. As shown in FIG. 1, the attachment of the slider 162 to the skirt portion 164 is by means of an inter-engaging annular bead and groove 166, 167 which snap-fit together. The assembly of nozzle 143 and valve stem 158 is urged into its normally closed position by spring 169 located between an inner surface of outer nozzle component 149 and valve cap 145.

The dispensing device 140 also includes a cap 170 which fits over the cartridge 141 enclosing the nozzle assembly 143 and includes a clip 171 similar to a pen cap.

In use of the device 140, the cap 170 is first removed. The device is then pre-loaded (as will be described below) to dispense a pre-selected quantity of medicament by selecting the required dose on the drive unit 10. The user then applies nozzle 143 to the point at which the medicament is to be introduced (for example, a nostril) and slides the slider 162 relative to the cartridge 141 thereby unseating the valve stem 158 from the valve seat 147 and allowing a portion of the medicament stored in the reservoir of the cartridge 141 to be dispensed.

The drive unit 10 of the dispensing device is located in housing 11. The end of housing 11 remote from nozzle 143 is closed off a cap 44. The housing 11 is open at the other end 61 for receiving the cartridge 141 as described above. A plunger 40 extends from end cap 44 towards piston member 22 and the drive unit 10 is operable to cause the plunger 40 to move axially towards nozzle 143 by a fixed distance determined by a dose selector (not shown) which is incorporated in end cap 44. The end cap 44 is rotatably mounted on the end of the housing 11 remote from plunger 40. Axial movement of the plunger 40 by said fixed distance causes the piston 22 to be urged to move the same distance to dispense the selected dose of the liquid medicament as described above.

The plunger 40 has an integrally formed thread 45 of a large lead angle. The plunger 40 slides axially within a plunger guide 43 which comprises inwardly directed teeth 49 which engage the thread 45 of the plunger 40. The plunger guide 43 is coupled to end cap 44 by co-operating bead and groove formations 54 and 53 which snap-fit together. The plunger guide 43 is rotatably fixed relative to the end cap 44 such that rotation of end cap 44 causes plunger guide 43 also to rotate. Rotation of plunger guide 43 in turn urges plunger 40 to move axially towards nozzle 143 by means of the engagement between teeth 49 and thread 45.

The plunger guide 43 comprises a number of circumferentially spaced ribs 51 which extend from an outer surface of the plunger guide 43 towards the inner wall of housing 11. The inner wall of housing 11 is provided with a matching number of T-shaped channels 52 in which the distal edges of ribs 51 are received. As shown in FIG. 1, the channels 52 extend axially from open end 61 to a point axially to the right of ribs 51 when in the position shown in FIG. 1.

A spring 48 is provided seated between an end face of the ribs 51 nearest of the end cap 44 and a spring seat 50 provided on an inwardly directed flange of the housing 11. The spring 48 acts to urge the plunger guide 43 towards the left as shown in FIG. 1.

The plunger guide 43 further comprises an end face 71 directed towards nozzle 143 which is provided with a series of teeth (not shown) which engage matching teeth on a ratchet disc 72 located between the plunger guide 43 and the cartridge 141. The ratchet disc 72 is rotationally fixed relative to cartridge 141. The matching sets of teeth are directed relative to one another such that relative rotation between the plunger guide 43 and the ratchet disc 72 is possible in only one direction.

In use to dispense a dose the end cap 44 is rotated which in turn rotates plunger guide 4.3 and also housing 11 by means of the engagement of the ribs 51 in the channels 52. The ratchet disc 72 and teeth on the end face 71 of the plunger guide 43 prevent rotation of the plunger guide 43 in the opposite direction. Rotation of the plunger guide 43 acts on the thread 45 of the plunger 40 through the teeth 49. Due to the incompressibility of the liquid medicament 21, the piston member 22 is unable to move towards the left as shown in FIG. 1 whilst the nozzle 143 is in the closed position. Hence, rotation of end cap 44 causes the plunger guide 43 and end cap 44 to move to the right as shown in FIG. 1 so as to accommodate the relative rotational movement between the thread 45 and teeth 49. As a result, the end cap 44, plunger guide 43 and ribs 51 move towards the right as shown in FIG. 1, compressing the spring 48 between the end face of ribs 51 and the spring seat 50 so as to charge the drive unit 10 with stored, spring energy. Co-operating formation 62 prevents housing 11 moving to the left as viewed in FIG. 1 under action of spring 48 as the spring force of spring 48 is insufficient to unseat the co-operating formation 62. An end face 55 of the T-shaped channels 52 acts as a limiter on the axial movement of the ribs 51 towards the right as shown in FIG. 1 so as to limit the energy stored in spring 48. Alternatively, the dose selector incorporated in end cap 44 may comprise means for limiting the rotation of end cap 44 during charging.

Once charged, the nozzle 143 is operated as described above by movement of the slider 162. At the point when the flow path to the outlet orifice 145 opens, the liquid medicament 21 is able to be dispensed, accommodating movement of the piston member to the left as shown in FIG. 1. At this point, spring 48 urges the ribs 51, plunger guide 43, dose selector 44 and plunger 40 to move to the left with piston member 22 to dispense the pre-selected dose of liquid medicament 21. The device is then ready to be re-charged for a subsequent actuation.

A problem has been found with dispensing liquid medicaments which are formed from suspensions using devices such as the one described above. It is a requirement of such devices that the liquid medicament 21 is stored without any air being present in the medicament cartridge 141 or medicament vial. This is due to the fact that the air can cause degradation of the medicament, because of the dangers of injecting air into the patient, especially where the medicament 21 is used intravenously, and also the inconsistency in shot weight which occurs when air is present. As a result, the vials or cartridges or other containers for the medicament 21 are formed with no air space. A problem has been found with dispensing suspensions from such vials or cartridges since it has been found that over time, the suspended element of the suspension settles out from the liquid carrier. In order for a correct dosage to be dispensed, it is therefore necessary to agitate the medicament to resuspend the medicament in the liquid carrier. This is not easy to achieve efficiently with many suspensions in a medicament vial or cartridge where there is no air space or other head space, especially where the user is elderly or infirm.

The present invention overcomes this problem by providing means for creating a vacuum or partial vacuum in the normally air free medicament vial or cartridge.

Referring to FIG. 1, a portion of the cartridge 141 is provided with an external screw thread on which is mounted a threaded nut 60 which abuts the open end 61 of the housing 11. In addition, end cap 44 is provided with a circumferential channel 63 in which is received the opposite end of the housing 11. In use, before operating the end cap 44 to charge the device for dispensation, the threaded nut 60 is rotated so as to move the threaded nut 60 to the right as viewed in FIG. 1 along the external thread of the cartridge 141. The movement of the threaded nut 60 to the right as shown in FIG. 1 forces the housing 11 and, by means of the engagement of the end of housing 11 in the circumferential channel 63, the end cap 44 to the right as viewed in FIG. 1. The rotation of the nut 60 provides sufficient force to unseat the co-operating formation 62 to allow the housing 11 to slide axially relative to the cartridge 141. The movement of the housing 11 in turn causes the piston member 22 to be moved to the right as viewed in FIG. 1 due to the engagement of the plunger 40, piston guide 43 and end cap 44. Consequently, operation of the threaded nut 60 produces an increase in volume of the cartridge and at the same time a reduction in pressure in the cartridge 141 below atmospheric since, with the nozzle 143 closed, the cartridge 141 is isolated from atmosphere. As such, a partial vacuum is created in the cartridge 141. The user then agitates the cartridge 141 by shaking the device to resuspend the suspension. The threaded nut 60 is then rotated in the opposite sense to return it to its original position. At the same time the suction effect of the partial vacuum in the cartridge 141 draws the piston member 22 back to its original position causing the co-operating formation 62 to re-engage. To aid re-engagement the faces of the co-operating formation contacting one another on re-engagement may be tapered so as to urge the formations to ride over one another.

Once the co-operating formations 62 are re-engaged, the device is ready to be charged and fired in the same manner as described above.

The present invention is not limited to the device described above which is provided as merely an illustrative example. The present invention provides equal application with both single dose and multiple dose devices. The medicament may be provided in a replaceable cartridge, vial or other container. Alternatively, the medicament may be provided in a chamber formed as part of the housing of the device, especially where the device is a single dose dispenser.

Other drive units may be used to power the dispensation of the medicament. One example is described in published European Patent application 0338806.

Other means of sealing the nozzle 143 and other nozzle types may be used without departing from the scope of the present invention.

The delivery system may be adapted for other types of administration, for example oral or sub-lingual.

1. A composition for treating skin ailments treatments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising: active agents and a carrier mixture, wherein said active agents are herbal extracts in a therapeutic effective dose, wherein said herbal extract is selected from the group consisting of mallow extract, rosemary extract and eucalyptus extract and said active agent mixes with carrier mixture for administration of said composition.

2. A composition as claimed in claim 1, consisting of the following ingredients in the following percentage range by weight:
(a) Water in the range of 40.19-49.12
(b) Sodium laureth sulphate in the range of 31.5-38.5
(c) Cocamide DEA in the range of 4.5-5.5
(d) Cocamidopropyl betaine in the range of 0.9-1.1
(e) Proylene glycol in the range of 0.27-0.33
(f) Citric acid in the range of 0.27-0.33
(g) Coal tar topical solution in the range of 5.4-6.6
(h) Sodium chloride in the range of 1.8-2.2
(i) Sodium layroyl sarcosinate in the range of 1.8-2.2
(j) Wheat germ oil in the range of 0.45-0.55
(k) Proteins (blends) in the range of 0.18-0.22
(l) Methylchloroisothiazolinone in the range of 0.045-0.045
(m) Mallow extract in the range of 0.9-1.1
(n) Rosemary oil in the range of 0.45-0.55
(o) Eucalyptus oil in the range of 0.45-0.55
(p) Carbomer in the range of 0.9-1.1.

3. A composition as claimed in claim 1 for treating skin ailments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex consisting of the following ingredients in the following percentage range by weight;
(a) Water in the range of 21.573-26.367
(b) Alcohol in the range of 63-77
(c) Sodium Iodide in the range of 2.7-3.3
(d) Potassium iodide in the range of 1.35-1.65
(e) Sodium thiosulphate in the range of 0.027-0.033
(f) Mallow extract in the range of 0.9-1.1
(g) Rosemary oil in the range of 0.225-0.27
(h) Eucalyptus oil in the range of 0.225-0.27.

4. A composition as claimed in claim 1 consisting of the following ingredients in the following percentage range by weight:
(a) Water in the range of 47.0205-57.4695
(b) Propylene glycol in the range of 3.6-4.4
(c) Mineral oil in the range of 2.7-3.3
(d) Cetyl alcohol in the range of 3.6-4.4
(e) Stearic acid in the range of 3.6-4.4
(f) Glycerol monostearate in the range of 2.7-3.3
(g) F D and C Yellow #5 solution in the range of 0.045-0.055
(h) D and C red #33 solution in the range of 0.0045-0.055
(i) Chamomile extract in the range of 0.9-1.1
(j) Carbomer in the range of 3.6-4.4
(k) Methyl paraben in the range of 0.225-0.275
(l) Propyl paraben in the range of 0.09-0.11
(m) Triethanolamine in the range of 0.54-0.66
(n) Milk amino acids in the range of 2.25-2.75
(o) Tea tree solution in the range of 2.7-3.3
(p) Mallow extract in the range of 0.9-1.1
(q) Coal tar topical solution in the range of 5.175-6.325
(r) Dimethicone in the range of 0.45-0.55
(s) Glycerine in the range of 2.7-3.3
(t) Petrolatum in the range of 0.9-1.1
(u) Cetearyl alcohol in the range of 2.25-2.75
(v) Ceteareth 20 in the range of 2.25-2.75
(w) Polysorbate 20 in the range of 1.8-2.2.

5. A composition as claimed in claim 1 consisting of at least 5% herbal extracts in an effective skin ailment treatment percentage mixed with a carrier mixture for topical administration to a human.

6. A composition as claimed in claim 1 for use in the treatment of skin ailments as wherein said herbal extract is least one percent mallow extract.

7. A system for use in skin ailment treatments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising a body wash composition, a spray composition and a cream composition, wherein said body wash composition, said spray composition and said cream composition further comprises an active agent and carrier mixture, wherein said active agents are herbal extracts in a therapeutic effective does, and said active agents mixes with said carrier mixture for administration of said body wash composition, said spray composition and said cream composition.

8. A system for use in skin ailment treatments as claimed in claim 7 wherein said body wash composition consisting of the following ingredients in the following range of proportions based on weight:
(a) Water in the range of 40.19-49.12
(b) Sodium laureth sulphate in the range of 31.5-38.5
(c) Cocamide DEA in the range of 4.5-5.5
(d) Cocamidopropyl betaine in the range of 0.9-1.1
(e) Proylene glycol in the range of 0.27-0.33
(f) Citric acid in the range of 0.27-0.33
(g) Coal tar topical solution in the range of 5.4-6.6
(h) Sodium chloride in the range of 1.8-2.2
(i) Sodium layroyl sarcosinate in the range of 1.8-2.2
(j) Wheat germ oil in the range of 0.45-0.55
(k) Proteins (blends) in the range of 0.18-0.22
(l) Methylchloroisothiazolinone in the range of 0.045-0.045
(m) Mallow extract in the range of 0.9-1.1
(n) Rosemary oil in the range of 0.45-0.55
(o) Eucalyptus oil in the range of 0.45-0.55
(p) Carbomer in the range of 0.9-1.1.

9. A system for use in skin ailment treatments as claimed in claim 7 wherein said spray composition consisting of the following ingredients in the following range of proportions based on weight:
(a) Water in the range of 21.573-26.367
(b) Alcohol in the range of 63-77
(c) Sodium Iodide in the range of 2.7-3.3
(d) Potassium iodide in the range of 1.35-1.65
(e) Sodium thiosulphate in the range of 0.027-0.033
(f) Mallow extract in the range of 0.9-1.1
(g) Rosemary oil in the range of 0.225-0.27
(h) Eucalyptus oil in the range of 0.225-0.27.

10. A system for use in skin ailment treatments as claimed in claim 7 wherein said cream composition consisting of the following ingredients in the following range of proportions:
(a) Water in the range of 47.0205-57.4695
(b) Propylene glycol in the range of 3.6-4.4
(c) Mineral oil in the range of 2.7-3.3
(d) Cetyl alcohol in the range of 3.6-4.4
(e) Stearic acid in the range of 3.6-4.4
(f) Glycerol monostearate in the range of 2.7-3.3
(g) F D and C Yellow #5 solution in the range of 0.045-0.055
(h) D and C red #33 solution in the range of 0.0045-0.055
(i) Chamomile extract in the range of 0.9-1.1
(j) Carbomer in the range of 3.6-4.4
(k) Methyl paraben in the range of 0.225-0.275
(l) Propyl paraben in the range of 0.09-0.11
(m) Triethanolamine in the range of 0.54-0.66
(n) Milk amino acids in the range of 2.25-2.75
(o) Tea tree solution in the range of 2.7-3.3
(p) Mallow extract in the range of 0.9-1.1
(q) Coal tar topical solution in the range of 5.175-6.325
(r) Dimethicone in the range of 0.45-0.55
(s) Glycerine in the range of 2.7-3.3
(t) Petrolatum in the range of 0.9-1.1
(u) Cetearyl alcohol in the range of 2.25-2.75
(v) Ceteareth 20 in the range of 2.25-2.75
(w) Polysorbate 20 in the range of 1.8-2.2.

11. A method of treating skin ailments selected from a group consisting of bruises, scars, cold sores, eczema, rosacea, shingles muscular joints and aches, moles, acne, rashes, insect bites, psoriasis, dry itch scaly skin, skin discoloration, blisters, burns, bee stings, athletes foot, boils, chapped hands and lips, cuts and scrapes and herpes simplex, comprising topically administering to a human having said skin ailment at least 1% herbal extracts, said herbal extracts being present in a topically acceptable pharmaceutical body wash composition.

12. A method as claimed in claim 11 wherein said topically acceptable pharmaceutical body wash composition consisting of the following ingredients in the following range of proportions:
(a) Water in the range of 40.19-49.12
(b) Sodium laureth sulphate in the range of 31.5-38.5
(c) Cocamide DEA in the range of 4.5-5.5
(d) Cocamidopropyl betaine in the range of 0.9-1.1
(e) Proylene glycol in the range of 0.27-0.33
(f) Citric acid in the range of 0.27-0.33
(g) Coal tar topical solution in the range of 5.4-6.6
(h) Sodium chloride in the range of 1.8-2.2
(i) Sodium layroyl sarcosinate in the range of 1.8-2.2
(j) Wheat germ oil in the range of 0.45-0:55
(k) Proteins (blends) in the range of 0.18-0.22
(l) Methylchloroisothiazolinone in the range of 0.045-0.045
(m) Mallow extract in the range of 0.9-1.1
(n) Rosemary oil in the range of 0.45-0.55
(o) Eucalyptus oil in the range of 0.45-0.55
(p) Carbomer in the range of 0.9-1.1.

13. A method treating skin ailments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising topically administering to a human having said skin ailment, at least 1% herbal extracts, said herbal extracts being present in a topically acceptable pharmaceutical spray composition.

14. A method of treating skin ailments as claimed in claim 13 wherein said topically acceptable pharmaceutical spray composition consisting of the following ingredients in the following range of proportions:
(a) Water in the range of 21.573-26.367
(b) Alcohol in the range of 63-77
(c) Sodium Iodide in the range of 2.7-3.3
(d) Potassium iodide in the range of 1.35-1.65
(e) Sodium thiosulphate in the range of 0.027-0.033
(f) Mallow extract in the range of 0.9-1.1
(g) Rosemary oil in the range of 0.225-0.27
(h) Eucalyptus oil in the range of 0.225-0.27.

15. A method of treating skin ailments selected from the group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising topically administering to a human having said skin ailment at least 1% herbal extracts, said herbal extracts being present in a topically acceptable pharmaceutical cream composition.

16. A method of treating skin ailments as claimed in claim 15 wherein said topically acceptable pharmaceutical cream composition consisting of the following ingredients in the following range of proportions:
(a) Water in the range of 47.0205-57.4695
(b) Propylene glycol In the range of 3.6-4.4
(c) Mineral oil in the range of 2.7-3.3
(d) Cetyl alcohol in the range of 3.6-4.4
(e) Stearic acid in the range of 3.6-4.4
(f) Glycerol monostearate in the range of 2.7-3.3
(g) F D and C Yellow #5 solution in the range of 0.045-0.055
(h) D and C red #33 solution in the range of 0.0045-0.055
(i) Chamomile extract in the range of 0.9-1.1
(j) Carbomer in the range of 3.6-4.4
(k) Methyl paraben in the range of 0.225-0.275
(l) Propyl paraben in the range of 0.09-0.11
(m) Triethanolamine in the range of 0.54-0.66
(n) Milk amino acids in the range of 2.25-2.75
(o) Tea tree solution in the range of 2.7-3.3
(p) Mallow extract in the range of 0.9-1.1
(q) Coal tar topical solution in the range of 5.175-6.325
(r) Dimethicone in the range of 0.45-0.55
(s) Glycerine in the range of 2.7-3.3
(t) Petrolatum in the range of 0.9-1.1
(u) Cetearyl alcohol in the range of 2.25-2.75
(v) Ceteareth 20 in the range of 2.25-2.75
(w) Polysorbate 20 in the range of 1.8-2.2.

17. A method for treating skin ailments selected from a group consisting of bruises, scars, cold sores, eczema, rosacea, shingles, muscular joints and aches, moles, acne, rashes, insect bites, psoriasis, dry itch scaly skin, skin discoloration, blisters, burns, bee stings, athletes foot, boils, chapped hands and lips, cuts and scrapes and herpes simplex, comprising topically administering to a human having said skin ailments at least 1% herbal extracts, said herbal extracts being present in a topically acceptable pharmaceutical body wash composition, spray composition and cream composition.

18. A method for treating skin ailments as claimed in claim 17 wherein said body wash composition consisting of the following ingredients in the following range of proportions:
(a) Water in the range of 40.19-49.12
(b) Sodium laureth sulphate in the range of 31.5-38.5
(c) Cocamide DEA in the range of 4.5-5.5
(d) Cocamidopropyl betaine in the range of 0.9-1.1
(e) Proylene glycol in the range of 0.27-0.33
(f) Citric acid in the range of 0.27-0.33
(g) Coal tar topical solution in the range of 5.4-6.6
(h) Sodium chloride in the range of 1.8-2.2
(i) Sodium layroyl sarcosinate in the range of 1.8-2.2
(j) Wheat germ oil in the range of 0.45-0.55
(k) Proteins (blends) in the range of 0.18-0.22
(l) Methylchloroisothiazolinone in the range of 0.045-0.045
(m) Mallow extract in the range of 0.9-1.1
(n) Rosemary oil in the range of 0.45-0.55
(o) Eucalyptus oil in the range of 0.45-0.55
(p) Carbomer in the range of 0.9-1.1.

19. A method for treating skin ailments as claimed in claim 18 wherein said spray composition consisting of the following ingredients in the following range of proportions;
(a) Water in the range of 21.573-26.367
(b) Alcohol in the range of 63-77
(c) Sodium Iodide in the range of 2.7-3.3
(d) Potassium iodide in the range of 1.35-1.65
(e) Sodium thiosulphate in the range of 0.027-0.033
(f) Mallow extract in the range of 0.9-1.1
(g) Rosemary oil in the range of 0.225-0.27
(h) Eucalyptus oil in the range of 0.225-0.27.

20. A method of treating skin ailments as claimed in claim 19, wherein said cream composition consisting of the following ingredients in the following range of proportions:
(a) Water in the range of 47.0205-57.4695
(b) Propylene glycol in the range of 3.6-4.4
(c) Mineral oil in the range of 2.7-3.3
(d) Cetyl alcohol in the range of 3.6-4.4
(e) Stearic acid in the range of 3.6-4.4
(f) Glycerol monostearate in the range of 2.7-3.3
(g) F D and C Yellow #5 solution in the range of 0.045-0.055
(h) D and C red #33 solution in the range of 0.0045-0.055
(i) Chamomile extract in the range of 0.9-1.1
(j) Carbomer in the range of 3.6-4.4
(k) Methyl paraben in the range of 0.225-0.275
(l) Propyl paraben in the range of 0.09-0.11
(m) Triethanolamine in the range of 0.54-0.66
(n) Milk amino acids in the range of 2.25-2.75
(o) Tea tree solution in the range of 2.7-3.3
(p) Mallow extract in the range of 0.9-1.1
(q) Coal tar topical solution in the range of 5.175-6.325
(r) Dimethicone in the range of 0.45-0.55
(s) Glycerine in the range of 2.7-3.3
(t) Petrolatum in the range of 0.9-1.1
(u) Cetearyl alcohol in the range of 2.25-2.75
(v) Ceteareth 20 in the range of 2.25-2.75
(w) Polysorbate 20 in the range of 1.8-2.2.
